(19) United States
(12) Patent Application Publication
(10) Pub. No.: US 2016/0369339 A1
(43) Pub. Date: Dec. 22, 2016

(54) BIOMARKER FOR PREECLAMPSIA

(71) Applicants: Cindy Anderson, Grand Forks, ND (US); Joyce Ohm, Grand Forks, ND (US)

(72) Inventors: Cindy Anderson, Grand Forks, ND (US); Joyce Ohm, Grand Forks, ND (US)

(21) Appl. No.: 14/902,244

(22) PCT Filed: Jun. 27, 2014
(86) PCT No.: PCT/US14/44673

§ 371 Date: Dec. 30, 2015

Related U.S. Application Data
(60) Provisional application No. 61/841,842, filed on Jul. 1, 2013.

(51) Int. Cl. C12Q 1/68

(52) U.S. Cl. CPC C12Q 1/6883; C12Q 2600/154

(57) ABSTRACT

The present invention provides a method to diagnose preeclampsia comprising measuring methylation level of at least one gene selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 in a first biological sample from a subject; and diagnosing preeclampsia in said subject based on a higher or lower methylation level in the first biological sample relative to the methylation level in a second biological sample from an individual without preeclampsia or control.

### CROSS-REFERENCE TO RELATED APPLICATIONS

The present application claims the priority benefits of U.S. Provisional Application No. 61/841,842, filed Jul. 1, 2013, which is expressly incorporated fully herein by reference.

### STATEMENT OF GOVERNMENT RIGHTS

This invention was made with the assistance of government support under Grant No EPS-081442 awarded by the NSF and Grant No. 5450-51000-41-00D awarded by USDA, ARS. The government has certain rights in the invention.

### BACKGROUND OF THE INVENTION

Preeclampsia is a syndrome unique to pregnancy characterized by onset hypertension and proteinuria in the second half of pregnancy (Fabry, Richart, Chengz, Van Bortel, & Staessen, 2010; Roberts, Pearson, Cutler, & Lindheimer, 2003). Affecting approximately 3% of pregnancies (Hutcheon, Lisonkova, & Joseph, 2011) and representing the 3rd leading cause of pregnancy-associated morbidity and mortality worldwide (Ghulmiyyah & Sibai, 2012), preeclampsia is associated with systemic vascular dysfunction and poor placental perfusion presenting later in pregnancy. However, the pathologic state of preeclampsia is thought to begin as a sub-clinical condition in the early weeks of gestation involving abnormal placentation, representing the etiology of later endothelial dysfunction, altered coagulation and a heightened inflammatory state. Early prevention, diagnosis, and treatment of preeclampsia are limited by the absence of reliable biomarkers to detect preeclampsia prior to manifestation of classic clinical symptoms (Conde-Agudelo, Villar, & Lindheimer, 2004; A. Gruslin & Lemyre, 2011a; A. Gruslin & Lemyre, 2011b).

Further underscoring the impact preeclampsia has on health outcomes is that a history of preeclampsia represents increased risk of developing cardiovascular disease later in life in women who survive preeclampsia (Barton & Sibai, 2008; Berends et al., 2008; Carty, Delles, & Dominiczak, 2010; Harskamp & Zeeman, 2007) and their offspring (Anderson, 2007; Geelhoed et al., 2010; Lampinen, Ronnback, Kaaja, & Groop, 2006; Mangos, 2006; Marin et al., 2000; Ray, Vermeulen, Schull, & Redelmeier, 2005; Roberts & Hubel, 2010; Smith, Pell, & Walsh, 2001; Staff, Dechend, & Pijnenborg, 2010; Wilson et al., 2003). Additionally, daughters of women with preeclampsia have a two-fold increased risk of developing preeclampsia during their own pregnancies (Skjaerven et al., 2005). Sons born to mothers with preeclampsia are also more likely to father a child who is a product of preeclampsia (Esplin et al., 2001), providing evidence of a genetic link to heritable preeclampsia risk. To date, no single candidate gene has been identified that offers clinical utility for identification of those at risk for preeclampsia or future sequelae.

### SUMMARY OF THE INVENTION

The present invention provides a method to diagnose preeclampsia in patient comprising measuring methylation level of at least one gene selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 in a first biological sample from a subject; and diagnosing preeclampsia in said subject based on a higher or lower methylation level in the first biological sample relative to the methylation level in a second biological sample from an individual without preeclampsia or control.

In one embodiment, the methylation level of at least two genes selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 and any combination thereof is measured. In another embodiment, the methylation level of at least 3 genes selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 and any combination thereof is measured. In another embodiment, the methylation level of at least 4 genes selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 and any combination thereof is measured. One embodiment provides that the methylation level of at least 5 genes selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 and any combination thereof, is measured. In one embodiment, the methylation level of at least 6, 7, 8, 9, 10, 11, 12 or 13 genes selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 and any combination thereof, is measured.

In one embodiment, an increased level of methylation of at least one gene selected from the group consisting of SERPINA9, SERPINA5 and PLEKHA2 indicates that the subject has or will develop preeclampsia. In another embodiment, a decreased level of methylation of least one gene selected from the group consisting of CPLX2, KIAA1609, MFAP2, CD80, KRT23, PKHD1, STMN2, RAP1A, UBE2G2 and KHDC1 indicates that the subject has or will develop preeclampsia.

In one embodiment, subject and individual are mammalian, such as a human. In one embodiment, the first and second samples comprise blood.

One embodiment provides for treating the subject for preeclampsia.

### BRIEF DESCRIPTION OF THE DRAWINGS

FIG. 1 depicts a bar graph demonstrating that blood pressure across three trimesters of pregnancy was not significantly different in women with preeclampsia and normotensive pregnancy; however, there was a significant increase in systolic and diastolic blood pressure on postpartum day 1.

FIGS. 2A-B demonstrate that a total of 207 CpG dinucleotides were identified as being differentially methylated in the maternal peripheral blood cells in women who developed preeclampsia as compared to those who were normotensive (FIG. 2A), including both gain and loss of methylation at individual CpG dinucleotides. 64% of the sites identified showed a gain of methylation in preeclampsia (FIG. 2B) while 36% of identified sites were associated with a loss of methylation (FIG. 2B).

FIG. 3 depicts differential methylation of top array sites.

FIG. 4: CpG dinucleotides identified as being differentially methylated in MPBCs (FIG. 2) were analyzed for methylation changes in placental tissue from women with preeclampsia as compared to normotensive controls (FIG. 4).

### DETAILED DESCRIPTION OF THE INVENTION

Epigenetic mechanisms of gene regulation are heritable and influenced by environmental factors, representing a potential mechanism and marker of disease and heritability of preeclampsia. DNA methylation of the linked cytosine and guanine (CpG) nucleotide bases is a major epigenetic event that can influence the regulation of gene expression in development, differentiation and aging, and is responsible for the maintenance of specific, heritable patterns of gene expression in humans (Jones & Baylin, 2007; Klose & Bird, 2006; Weber, Stresemann, Brueckner, & Lyko, 2007). These CpG sites may be located within the gene promoter, gene body, or flanking island, shelf or shore regions (Irizarry, Wu, & Feinberg, 2009). Although most studies of DNA methylation have focused on the promoter regions, recent evidence suggests that the altered DNA methylation in the CpG island shore regions are strongly related to gene expression (Irizarry et al., 2009).

Most DNA methylation patterns are established in utero though cells may continue to remodel chromatin and establish new expression patterns during postnatal development (Lahiri, Maloney, & Zawia, 2009; Loke et al., 2012; Zawia, Lahiri, & Cardozo-Pelaez, 2009). Methylation patterns established during fetal life may be affected by placental insufficiency, limiting the utilization and availability of key nutrients, including those impacting methylation (e.g., folate, methionine, cysteine). Recent reports suggest global DNA methylation is increased in placenta tissue samples of patients with preeclampsia (Jia et al., 2012; Kulkarni, Chavan-Gautam, Mehendale, Yadav, & Joshi, 2011), and hypomethylation in promoter regions of multiple genes may be an indicator and/or consequence of early-onset preeclampsia (Yuen, Penaherrera, von Dadelszen, McFadden, & Robinson, 2010). To date, no published reports of differential DNA methylation in preeclampsia linking mother and offspring are available, limiting evidence to correlate the fetal epigenome with the maternal intrauterine environment.

In addition to the impact on short-term gene expression, epidemiological evidence suggests that environmental exposures during development may play a role in disease susceptibility later in life, and researchers have hypothesized that epigenetic changes in gene regulation are responsible for this phenomenon (Herceg, 2007; Jirtle & Skinner, 2007). As DNA methylation patterns are largely established during fetal life, epigenomic alterations represent a plausible explanation of the heritable development of preeclampsia and cardiovascular disease resulting from placental insufficiency. Identified herein are distinct DNA methylation patterns in women during early pregnancy that can predict preeclampsia and in fetal-derived placental tissue that can represent heritable changes in the epigenome established during fetal life.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

Preeclampsia can be defined/diagnosed by persistent high blood pressure that develops during pregnancy or during the postpartum period that is associated with a lot/increased protein in the urine or the new development of decreased blood platelets, trouble with the kidney or liver, fluid in the lungs, or signs of brain trouble such as seizures and/or visual disturbances.

The articles “a” and “an” are used herein to refer to one or to more than one (i.e. to at least one) of the grammatical object of the article. By way of example, “an element” means one element or more than one element.

“Plurality” means at least two.

A “subject” or “patient” is a vertebrate, including a mammal, such as a human. Mammals include, but are not limited to, humans, farm animals, sport animals and pets.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The term “gene” refers to a nucleic acid sequence that comprises control and coding sequences necessary for producing a polypeptide or precursor. The polypeptide may be encoded by a full length coding sequence or by any portion of the coding sequence. The gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns.

The term “gene expression” refers to the process by which a nucleic acid sequence undergoes successful transcription and/or translation such that detectable levels of the nucleotide sequence are expressed.

The term “methylation level” refers to the state of methylation of a genomic sequence, refers to the characteristics of a DNA segment at a particular genomic locus relevant to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles. The term “methylation” or “methylation level” also refers to the relative or absolute concentration of methylated C or unmethylated C at any particular stretch of residues of a gene in a biological sample.

An “increase” or a “decrease” refers to a detectable positive or negative change in quantity from a standard/control. Other terms indicating quantitative changes or differences from a comparative basis, such as “more” or “less,” are used in this application in the same fashion as described above.

“Standard” or “control” as used herein refers to a sample comprising a genomic sequence of a predetermined amount or methylation level (which may include multiple different and separable characteristics related to methylation) suitable for the use of a method of the present invention, in order for comparing the amount or methylation level of a particular genomic sequence that is present in a test sample from a subject. A sample serving as a standard or control provides an average amount or methylation level of a gene of interest that is typical for a defined time (e.g., first trimester) during pregnancy in the blood of an average, healthy pregnant woman carrying a normal fetus, both of who are not at risk of developing any pregnancy-associated disorders or complications.

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. Furthermore, the term “nucleic acid sequences” contemplates the complementary sequence and specifically includes any nucleic acid sequence that is substantially homologous to the both the nucleic acid sequence and its complement.

The term “nucleic acid” or “polynucleotide” refers to deoxyribonucleic acids (DNA) or ribonucleic acids (RNA) and polymers thereof in either single-or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma -carboxyglutamate, and O-phosphoserine.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

The term “biological sample” refers to a sample obtained from an organism (e.g., a human patient) or from components (e.g., cells) of an organism. The sample may be of any biological tissue or fluid. The sample may be a “clinical sample” which is a sample derived from a patient. Such samples include, but are not limited to, sputum, blood, blood cells (e.g., white cells), amniotic fluid, plasma, semen, bone marrow, circulating tumor cells, circulating DNA, circulating exosomes, and tissue or fine needle biopsy samples, urine, peritoneal fluid, and pleural fluid, or cells therefrom. Biological samples may also include sections of tissues such as frozen sections or formalin fixed paraffin embedded sections aken for histological purposes. A biological sample may also be referred to as a “patient sample.”

As used herein, “health care provider” includes either an individual or an institution that provides preventive, curative, promotional or rehabilitative health care services to a subject, such as a patient. In one embodiment, the data is provided to a health care provider so that they may use it in their diagnosis/treatment of the patient.

The terms “comprises”, “comprising”, and the like can have the meaning ascribed to them in U.S. Patent Law and can mean “includes”, “including” and the like. As used herein, “including” or “includes” or the like means including, without limitation.

### Gene Methylation (Markers)

The determination of the methylation level/state of a gene can be measured by methods available and known to an art worker. For example, DNA methylation can be detected by the following, but not limited to, assays:

Methylation-Specific PCR (MSP), which is based on a chemical reaction of sodium bisulfite with DNA that converts unmethylated cytosines of CpG dinucleotides to uracil or UpG, followed by traditional PCR. However, methylated cytosines will not be converted in this process, and primers are designed to overlap the CpG site of interest, which allows one to determine methylation status as methylated or unmethylated.

Whole genome bisulfite sequencing, also known as BS-Seq, which is a high-throughput genome-wide analysis of DNA methylation. It is based on aforementioned sodium bisulfite conversion of genomic DNA, which is then sequenced on a Next-generation sequencing platform. The sequences obtained are then re-aligned to the reference genome to determine methylation states of CpG dinucleotides based on mismatches resulting from the conversion of unmethylated cytosines into uracil.

The HELP assay, which is based on restriction enzymes' differential ability to recognize and cleave methylated and unmethylated CpG DNA sites.

ChIP-on-chip assays, which is based on the ability of commercially prepared antibodies to bind to DNA methylation-associated proteins like MeCP2.

Restriction landmark genomic scanning, an assay based upon restriction enzymes' differential recognition of methylated and unmethylated CpG sites; the assay is similar in concept to the HELP assay.

Methylated DNA immunoprecipitation (MeDIP), analogous to chromatin immunoprecipitation, immunoprecipitation is used to isolate methylated DNA fragments for input into DNA detection methods such as DNA microarrays (MeDIP-chip) or DNA sequencing (MeDIP-seq).

Pyrosequencing of bisulfate treated DNA. This is sequencing of an amplicon made by a normal forward primer but a biotinylated reverse primer to PCR the gene of choice. The Pyrosequencer then analyses the sample by denaturing the DNA and adding one nucleotide at a time to the mix according to a sequence given by the user. If there is a mis-match, it is recorded and the percentage of DNA for which the mis-match is present is noted. This gives the user a percentage methylation per CpG island.

Molecular break light assay for DNA adenine methyltransferase activity—an assay that relies on the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase.

Methyl Sensitive Southern Blotting is similar to the HELP assay, although uses Southern blotting techniques to probe gene-specific differences in methylation using restriction digests. This technique is used to evaluate local methylation near the binding site for the probe.

MethylCpG Binding Proteins (MBPs) and fusion proteins containing just the Methyl Binding Domain (MBD) are used to separate native DNA into methylated and unmethylated fractions. The percentage methylation of individual CpG islands can be determined by quantifying the amount of the target in each fraction. Very sensitive detection can be achieved in FFPE tissues with Abscription based detection.

Differentially methylated regions (DMRs), as genomic regions with different methylation statuses among multiple samples (tissues, cells, individuals or others), are can be functional regions involved in gene transcriptional regulation etc. The identification of DMRs among multiple tissues (T-DMRs) provides a comprehensive survey of epigenetic differences among human tissues. DMRs between cancer and normal samples (C-DMRs) demonstrate the aberrant methylation in cancers. It is well known that DNA methylation is associated with cell differentiation and proliferation.

QDMR (Quantitative Differentially Methylated Regions) is a quantitative approach to quantify methylation difference and identify DMRs from genome-wide methylation profiles by adapting Shannon entropy (http://bioinfo.hrbmu.edu.cn/qdmr) The platform-free and species-free nature of QDMR makes it potentially applicable to various methylation data. This approach provides an effective tool for the high-throughput identification of the functional regions involved in epigenetic regulation. QDMR can be used as an effective tool for the quantification of methylation difference and identification of DMRs across multiple samples.

The methylation level of certain genes has been demonstrated herein to be predictive of preeclampsia. These genes include the following (or those homologous thereto):

The CPLX2 gene codes for complexin-2 protein in humans. Proteins encoded by the complexin/synaphin gene family are cytosolic proteins that function in synaptic vesicle exocytosis. These proteins bind syntaxin, part of the SNAP receptor. The protein product of this gene binds to the SNAP receptor complex and disrupts it, allowing transmitter release. Transcript variants encoding the same protein have been found for this gene. The accession number for the gene is UCSC RefGene Accession NM_001008220; NM_006650. The following probe sequence corresponds to a portion of the gene sequence and includes the individual CpG that is gaining or losing methylation—Allele A Probe Sequence

(SEQ ID NO: 1)
TAAAACTCCTACAAATCTTTCACAAACCTTATACAAAACCCCTCCTCTCC.

The KIAA1609 gene codes for TBC/LysM-associated domain containing 1 (TLDC1) protein. The accession number for the gene is UCSC RefGene Accession NM_020947. The following probe sequence corresponds to a portion of the gene sequence and includes the individual CpG that is gaining or losing methylation—Allele A Probe Sequence

(SEQ ID NO: 2)
ATCACATCAAAATTCTTCAAAACAAACCRTATAACCCCCAATTTTCAAAC.

The MFAP2 gene codes for Microfibrillar-associated protein 2 protein in humans. Microfibrillar-associated protein 2 is a major antigen of elastin-associated microfibrils and a candidate for involvement in the etiology of inherited connective tissue diseases. This gene encodes two transcripts with two alternatively spliced 5′ untranslated exons. These two transcripts contain the same 8 coding exons, and therefore, encode the same protein. The accession number for the gene is UCSC RefGene Accession NM_002403.

The CD80 gene codes for Cluster of Differentiation 80 (also CD80 and B7-1) which is a protein found on activated B cells and monocytes that provides a costimulatory signal necessary for T cell activation and survival. It is the ligand for two different proteins on the T cell surface: CD28 (for autoregulation and intercellular association) and CTLA-4 (for attenuation of regulation and cellular disassociation). CD80 works with CD86 to prime T cells. The accession number for the gene is UCSC RefGene Accession NM_005191. The following probe sequence corresponds to a portion of the gene sequence and includes the individual CpG that is gaining or losing methylation - Allele A Probe Sequence

(SEQ ID NO: 3)
TATTTACACAAATAAAACCTAACAACACCTTACATAAATTACAATAAACC.

The KRT23 gene codes for Keratin, type I cytoskeletal 23 protein in humans. The protein encoded by this gene is a member of the keratin family. The keratins are intermediate filament proteins responsible for the structural integrity of epithelial cells and are subdivided into cytokeratins and hair keratins. The type I cytokeratins consist of acidic proteins which are arranged in pairs of heterotypic keratin chains. The type I cytokeratin genes are clustered in a region of chromosome 17q12-q21. The accession number for the gene is UCSC RefGene Accession N_015515. The following probe sequence corresponds to a portion of the gene sequence and includes the individual CpG that is gaining or losing methylation—Allele A Probe Sequence

(SEQ ID NO: 4)
TTCRCATATACAATATAATTAAACACATAAATTCATAAATAACACTACCC.

The PKHD1 gene codes for a human protein. The accession number for the gene is UCSC RefGene NM_138694; NM_170724. The following probe sequence corresponds to a portion of the gene sequence and includes the individual CpG that is gaining or losing methylation—Allele A Probe Sequence

(SEQ ID NO: 5)
CATTCTAATTCACATCCCCCAATTCCTAATCATATTTATCTACRTCTAAC.

The STMN2 gene codes for Stathmin-2 is a protein in humans. The accession number for the gene is UCSC RefGene Accession NM_007029. The following probe sequence corresponds to a portion of the gene sequence and includes the individual CpG that is gaining or losing methylation—Allele A Probe Sequence

(SEQ ID NO: 6)
AAAACCTATTCAAAAACTCAAAAACATTTACCTTCCAAAATTATTCTATC.

The RAP1A gene codes for Ras-related protein Rap-1A in humans. The product of this gene belongs to the family of Ras-related proteins. These proteins share approximately 50% amino acid identity with the classical RAS proteins and have numerous structural features in common. The most striking difference between RAP proteins and RAS proteins resides in their 61st amino acid: glutamine in RAS is replaced by threonine in RAP proteins. The accession number for the gene is UCSC RefGene Accession NM_001010935; NM_002884. The following probe sequence corresponds to a portion of the gene sequence and includes the individual CpG that is gaining or losing methylation—Allele A Probe Sequence

(SEQ ID NO: 7)
CATCAACAAAACTAACTTTTAACAACATAAATCAAATACRATCATTCCTC.

The UBE2G2 gene codes for Ubiquitin-conjugating enzyme E2 G2 protein in humans. The modification of proteins with ubiquitin is a cellular mechanism for targeting abnormal or short-lived proteins for degradation. Ubiquitination involves at least three classes of enzymes: ubiquitin-activating enzymes, or Els, ubiquitin-conjugating enzymes, or E2s, and ubiquitin-protein ligases, or E3s. This gene encodes a member of the E2 ubiquitin-conjugating enzyme family. This gene is ubiquitously expressed, with high expression seen in adult muscle. Ube2g2 is known to interact with a variety of other proteins, including but not limited to ubiquitin, the E3 gp78, and the Hrdl RING. The accession number for the gene is UCSC RefGene Accession NM_182688; NM_003343. The following probe sequence corresponds to a portion of the gene sequence and includes the individual CpG that is gaining or losing methylation—Allele A Probe Sequence

(SEQ ID NO: 8)
RCCACCTAATATCTACTAAAACAAAACATAAAAACACACCTCAATTTCCC.

The KHDC1 codes for a protein expressed in humans. The accession number for the gene is UCSC RefGene Accession NM_030568. The following probe sequence corresponds to a portion of the gene sequence and includes the individual CpG that is gaining or losing methylation—Allele A Probe Sequence

(SEQ ID NO: 9)
TTAAATTTTTTATAAAAACAAATTCTCCCTCTATTACCCCAACTAATTTC.

The SERPINA9 gene codes for Serpin A9, also known as centerin or GCET1, protein in humans and is located on chromosome 14q32.1. Serpin A9 is a member of the serpin family of serine protease inhibitors. SERPINA9 is expressed in germinal center B cells and lymphoid malignancies. SERPINA9 is likely to function in vivo as an inhibitor of trypsin-like proteases/serpin peptidase inhibitor, clade A (alpha-1 antiproteinase, antitrypsin), member 9. The accession number for the gene is UCSC RefGene Accession NM_175739; NM_001042518. The following probe sequence corresponds to a portion of the gene sequence and includes the individual CpG that is gaining or losing methylation—Allele A Probe Sequence

(SEQ ID NO: 10)
RTTTCCCAAAATAAACACTAAAATCTTAACTTACATTACCTAACCTCTAC.

The SERPINA5 gene codes for Protein C inhibitor (PCI, SERPINA5), a serine protease inhibitor (serpin) which limits the expression of protein C (an anticoagulant)/serpin peptidase inhibitor, clade A (alpha-1 antiproteinase, antitrypsin), member 5. The accession number for the gene is UCSC RefGene Accession NM_000624. The following probe sequence corresponds to a portion of the gene sequence and includes the individual CpG that is gaining or losing methylation—Allele A Probe Sequence

(SEQ ID NO: 11)
ATTTATTCACACTTTTAAAAAAACCACCTACTACACCAAATACTTTAAAC.

The PLEKHA2 gene codes for Pleckstrin homology domain-containing family A member 2 protein in humans. The accession number for the gene is UCSC RefGene Accession NM_021623. The following probe sequences correspond to portions of the gene sequence and includes the individual CpG that is gaining or losing methylation - Allele A Probe Sequence

(SEQ ID NO: 12)
AATAACCAAAATCACCACCACCAAAAATCAAACTACAAAAAAT
AAAACCA;;

### Allele B Probe Sequence

(SEQ ID NO: 13)
AATAACCAAAATCGCCGCCGCCAAAAATCGAACTACGAAAAATAAAACCG.

Any sequences related to the sequences listed above and available to an art worker, e.g., homologous sequences, are useful in the methods of the invention as well. For example, sequences for use in the invention have at least about 50% or about 60% or about 70%, about 71%, about 72%, about 73%, about 74%, about 75%, about 76%, about 77%, about 78%, or about 79%, or at least about 80%, about 81%, about 82%, about 83%, about 84%, about 85%, about 86%, about 87%, about 88%, or about 89%, or at least about 90%, about 91%, about 92%, about 93%, or about 94%, or at least about 95%, about 96%, about 97%, about 98%, or about 99% sequence identity compared to the accession numbers/sequences provided herein and/or any other such sequence available to an art worker, using one of alignment programs available in the art using standard parameters. In another embodiment, the DNA sequence has at least 80% , including at lesat 95%, sequence identity with the sequences disclosed herein.

Methods of alignment of sequences for comparison are available in the art. Thus, the determination of percent identity between any two sequences can be accomplished using a mathematical algorithm. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

### EXAMPLES

The following examples are provided in order to demonstrate and further illustrate certain embodiments and aspects of the present invention and are not to be construed as limiting the scope thereof.

### Example I

### Materials and Methods

### Participant Recruitment and Sample Collection

In this study, a convenience sample of nulliparous women in the first trimester of pregnancy was recruited from a local community. Eligibility criteria included no previous deliveries, age >18 years and pregnancy less than 14 weeks gestation. Participants were provided study details from research team members and enrolled upon consent. Approval from the Institutional Review Boards of the University of North Dakota and the cooperating Health System were received and human subjects protection assured throughout the study. Maternal peripheral blood (MP-B) was collected from participants via venipuncture in the first trimester of pregnancy. White blood cells were isolated, providing the source for the

DNA used to quantify methylation in this study. Upon delivery, placental tissue was collected for analysis of DNA methylation and placed in cold physiological saline solution. After removal of decidua, chorionic tissue was collected from six individual sites equally spaced along the placental periphery in morphologically normal areas and stored at −80° C. until analyses. Samples obtained from the 12:00 position were homogenized for analyses. Pregnancy outcome was determined by medical record abstraction. Preeclampsia was identified by documented diagnosis or evidence of new-onset hypertension (systolic blood pressure >140 mmHg or diastolic blood pressure >90 mmHg) combined with proteinuria (+1 single sample or >300 mg/24 hour urine sample) during the second half of pregnancy or during the postpartum hospitalization (Roberts et al., 2003). Achievement of diagnostic criteria in relation to gestational age determined subcategorization of early (<34 weeks) versus late onset preeclampsia (>34 weeks)(Hogg, Blair, von Dadelszen, & Robinson, 2013; Kucukgoz Gulec et al., 2013). Women with systolic blood pressure of <140 mmHg, diastolic blood pressure of <90 mmHg and absence of significant proteinuria (<trace in single sample) were categorized as normotensive. Blood pressure was determined based on the highest blood pressure recorded on at least two occasions after 20 weeks of pregnancy.

### Genome-Wide DNA Methylation

DNA was purified from maternal peripheral blood and placental tissue with phenol-chloroformisoamyl alcohol (Life Technologies Corporation) using established isolation procedures (Ohm et al., 2010). Approximately 3 ug of total genomic DNA was sent to the Illumina certified Biomedical Genomics Core at the University of Minnesota for Illumina Infinium DNA methylation 450K bead-based array analysis (Bibikova et al., 2011; Dedeurwaerder et al., 2011; Sandoval et al., 2011). Genome-wide DNA methylation data were normalized and differentially methylated CpG sites were identified using the GenomeStudio DNA methylation module (Illumina) Average beta scores for each individual CpG site were compiled for normotensive control and preeclampsia samples (n=6/group). The Infinium platform uses a bead-based array to identify the percent methylation at any single CpG dinucleotide loci. Average beta scores were assigned based on the percentage of methylation at individual loci within a sample and ranged from 0 (0% methylation) to 1 (100% methylation) within a given sample. Sites without measureable beta scores across any of the 12 samples were discarded. Individual CpG dinucleotides with significantly increased methylation were defined as having a change in beta (delta-beta) score in preeclampsia compared to normotensive control greater than 0.2 (indicating >20% increase in methylation compared to controls) and a p-value <0.05 by two-tailed T Test. CpG dinucleotides with significantly decreased methylation had a delta-beta across preeclampsia samples of <-0.2 (indicating >20% decrease in methylation compared to normotensive controls) and a p-value <0.05. Samples were also independently analyzed using the NIMBL (Numerical Identification of Methylation Biomarker Lists) Infinium analysis package for Matlab provided by Frank Wessely, School of Veterinary Medicine and Science, University of Nottingham, UK. The NIMBL package is publically available and requires Matlab (tested using Matlab release version 7.11) and the Statistics and Bioinformatics Matlab tool boxes. NIMBL has been specifically designed to identify biomarkers in clinical samples by identifying spots with maximum absolute distance between control and experimental samples, taking into account the heterogeneity that is common in clinical samples (Wessely & Emes, 2012; Wessely & Emes, 2012). NIMBL and NIMBL-qc were performed on all maternal white blood samples. The top 20 differentially methylated sites were identified with a maximum of two preeclampsia samples masked to allow for heterogeneity.

### Results

### Participant Demographics

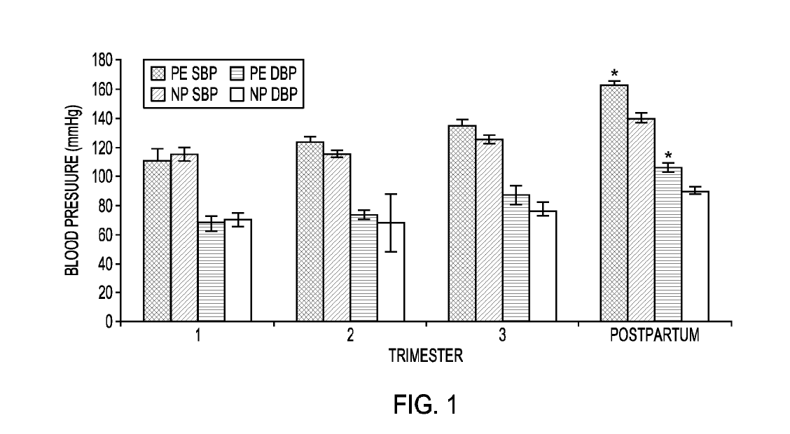

A total of 64 women were enrolled in the study, with 86% (n=55) retained through the duration of the study. Nine women did not complete the study due to moving from the area (n=2), miscarriage (n=2), changed to non-participating provider (n=1) and lost to follow up (n=4). Of the women who completed the study, 12.7% (n=7) developed preeclampsia. First trimester maternal white blood cell (average gestational age of 64.5 days across all samples) and placenta samples were available for 6 of the women with preeclampsia and were included in the DNA methylation analysis, matched with 6 women with normotensive pregnancy outcomes based upon maternal age (range 0-9 years in difference) and weight at first prenatal visit (range 1-20 lbs difference). Demographic data describing study participants included in DNA methylation analyses are shown in Table 1. Gestational weight gain was significantly greater among women who developed preeclampsia (p=0.038) though there were no significant group differences in other weight measures in mothers or infants. Blood pressure across three trimesters of pregnancy was not significantly different in women with preeclampsia and normotensive pregnancy, however, there was a significant increase in systolic and diastolic blood pressure on postpartum day 1 (FIG. 1). The timing of hypertension onset, coupled with absence of significant differences in pregnancy duration and infant birth weight, indicates the more common syndrome of late onset preeclampsia.

TABLE 1
Sample Characteristics

| | Preeclampsia | Normotensive Pregnancy |
|---|---|---|
| Variable | (n = 6) | (n = 6) |
| Maternal age at delivery (years) | 22.78 (1.44) | 27.5 (3.65) |
| Race: White, Non-Hispanic | 6 (100) | 6 (100) |
| Gestational age at first prenatal visit (days from | 70 (5.4) | 59 (7.7) |
| LMP) | | |
| Gestational age at first trimester WBC | 11.86 (0.60) | 11.98 (0.54) |
| Collection (weeks) | | |
| Gestational age at delivery (weeks) | 38.4 | 40 |
| Weight at first prenatal visit (lbs) | 180.9 (12.65) | 176.4 (12.23) |
| Weight at delivery (lbs) | 223.5 (12.27) | 201.0 (9.11) |
| Gestational weight gain (lbs) | 42.6 (5.49)* | 24.53 (5.17) |
| Infant birth weight | 3299 (177.9) | 3212.5 (260.7) |
| Infant gender, N | | |
| Female | 5 | 3 |
| Male | 1 | 3 |
| Delivery Type | | |
| Vaginal | 3 | 5 |
| Vacuum-assist | 2 | 0 |
| C-Section | 1 | 1 |

Data reported as mean (SEM)
*= p < 0.05

### Identification of DNA Methylation Biomarkers in Maternal Peripheral Blood

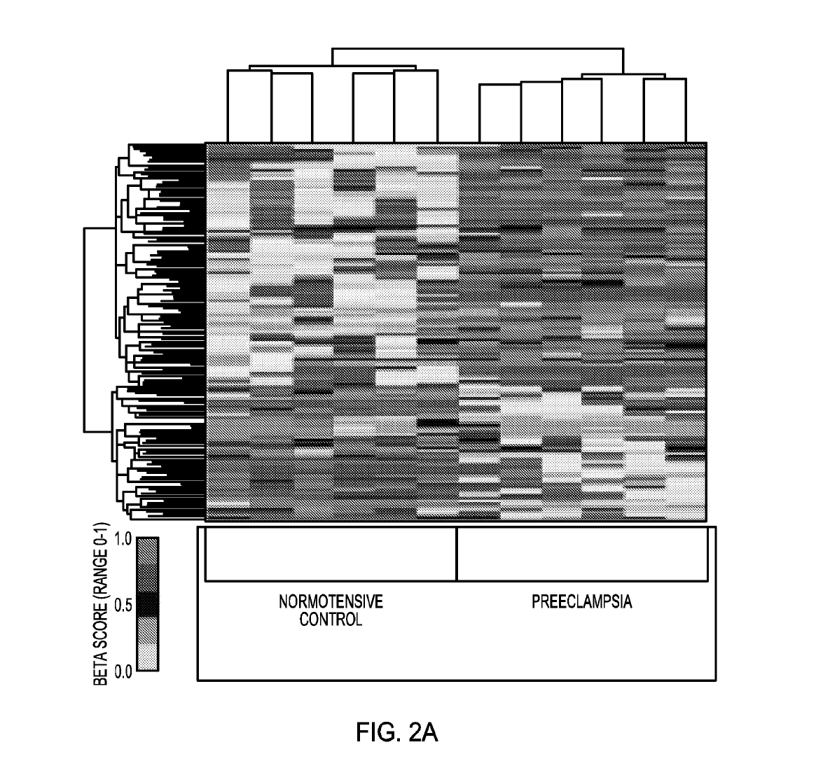

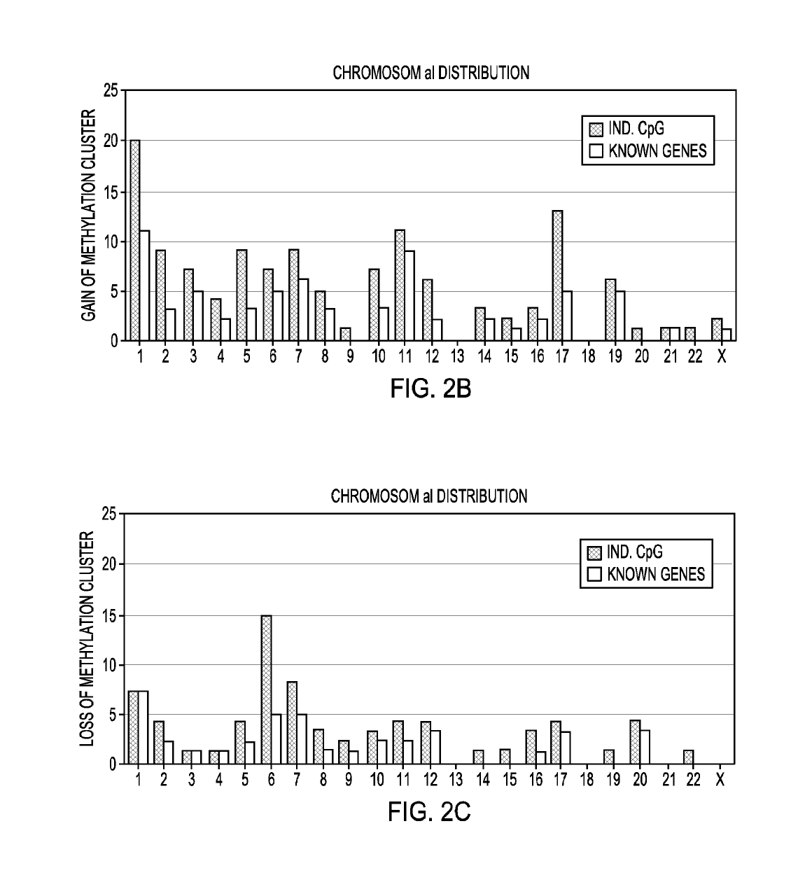

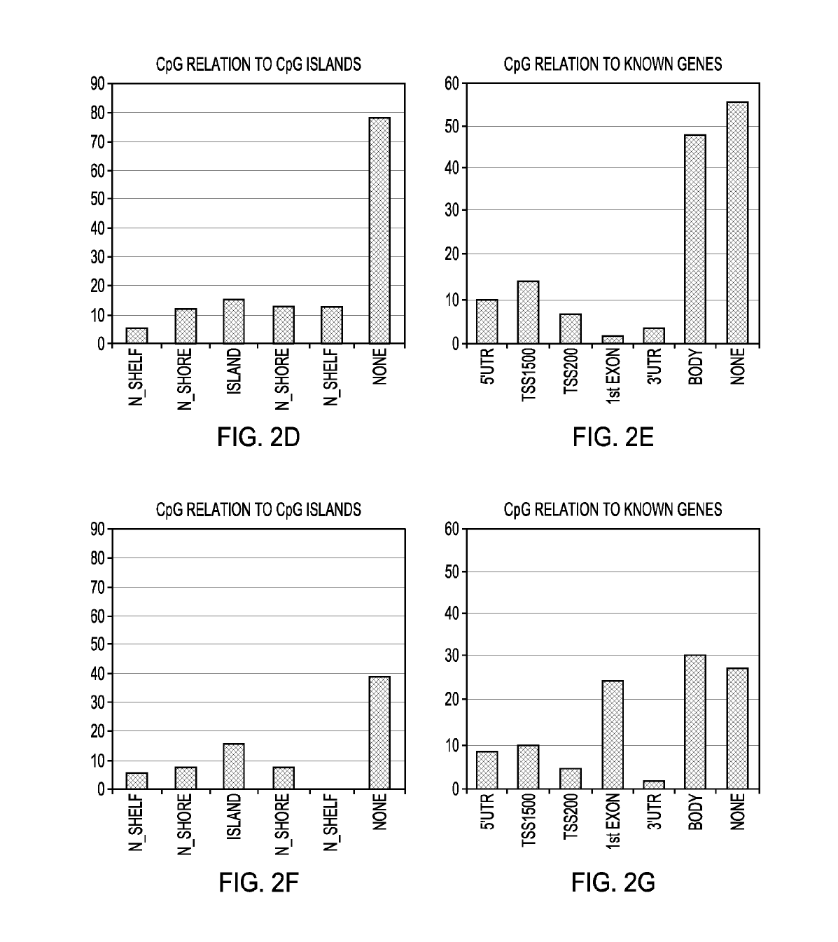

A total of 207 CpG dinucleotides were identified as being differentially methylated in the maternal peripheral blood cells in women who developed preeclampsia as compared to those who were normotensive (FIG. 2A), including both gain and loss of methylation at individual CpG dinucleotides. 64% of the sites identified showed a gain of methylation in preeclampsia (FIG. 2B) while 36% of identified sites were associated with a loss of methylation (FIG. 2B). For sites either gaining or losing methylation, chromosomal distribution of these CpG dinucleotides was seen across most chromosomes in the genome (FIG. 2B). The majority of these differentially methylated sites were not associated with known CpG islands (FIG. 2B), and were found to be either within the body of known genes or in areas of the genome that are not currently known to be associated with any annotated gene (FIG. 2B). The remainder of differentially methylated CpG site were found in CpG islands or flanking shore and shelf regions (Doi et al., 2009) (FIG. 2B). Biological pathways in genes differentially methylated in preeclampsia were determined by cluster analysis (Table 2). Genes with significant gain in methylation were associated with cell signal transduction involving lipid binding, protease enzyme inhibition, protein-protein interaction, cell cycle processes and adhesion. Signaling pathways involving cellular metabolic processes predominated in genes that had significant methylation loss.

TABLE 2
Biological Pathways Associated with Differentially Methylated Genes in Preeclampsia

| | | | Enrichment | |
|---|---|---|---|---|
| | Functional Cluster | | Score | Genes |
| Methylation Gain | 1 | Pleckstrin homology | 3.18 | ABR, OSBPLS, PLCH1, PLEKHA2, PLEKHA7, SPTBN4, TEC |
| | 2 | Protease enzyme inhibition | 1.12 | COLGA3, SERPINA9, SERPINB9 |
| | 3 | Ribonucleotide binding | 0.78 | ABC6, DHX37, MOV10, GNA12, LOC100133091, |
| | | | | NUBP1, PFKP, RPS6KA2, SEPT9, STK32C, TEC |
| | 4 | Ankyrin repeat | 0.71 | BCOR, ANKRD34B, MIB2 |
| | 5 | Cell cycle processes | 0.63 | MAD1L1, USH1C, PRM1, PSMB9 |
| | 6 | Cell adhesion | 0.53 | ASTN1, COL6A3, ITGB2, LAMB1 |
| | 7 | Actin binding | 0.47 | MIB2, MYRIP, SPTBN4 |
| | 8 | Phosphate metabolic | 0.34 | PFKP, RPS6KA2, TEC |
| | | processing | | |
| | 9 | Ion binding | 0.28 | FOXK2, MAN281, MMEL1, MIB2, MYRIP, NUBP1, PFKP, PLCH1, |
| | | | | RPS6KA2, STK32C, TEC, ZNF490, ZNF664 |
| | 10 | Neurologic processing: | 0.16 | USH1C, MAN281, OR5H15, SPTBN4 |
| | | cognition, perception | | |
| Methylation Loss | 1 | Membrane/cell fraction | 2.77 | ABCA1, GNAS, TAPBP, DYNLL1, ORPD1, |
| | | | | PRKCZ, SLC23A2, STMN2 |
| | 2 | Phosphate metabolic | 1.9 | CD80, DYNLL1, PHXD1, PRKCZ, PRKAR1B |
| | | processing | | |
| | 3 | cAMP and cyclic nucleotide | 1.74 | ABCA1, GNAS, OPRD1 |
| | | biosynthetic and metabolic | | |
| | | processes | | |
| | 4 | Nucleotide binding | 1.68 | AAK1, ABCA1, GNAS, GIMAP1, DNAH9, |
| | | | | MAPK10, PRKCZ, PRKAR1B, MGC87042, SFRS9, TUBAL3 |
| | 5 | Protein amino acid | 1.13 | AAK1, ERC1, MAPK10, PRKCZ, |
| | | phosphorylation | | PRKAR1B |
| | 6 | Protein kinase activity | 1.08 | PKHD1, PRKCZ, PRKAR1B |
| | 7 | Protein | 0.99 | CD80, PRKCZ, PRKAR1B |
| | | modification/metabolism | | |
| | 8 | Nucleoside binding | 0.91 | AAK1, ABCA1, DNAH9, MAPK10, PRKC2, PRKAR1B, MGC87042 |
| | 9 | Cellular | 0.88 | ABCA1, CD80, PRDM16, PCBD1 |
| | | biosynthetic/metabolic | | |
| | | processes | | |
| | 10 | Transcription cofactor | 0.81 | PRDM16, BRDT, PCBD1 |
| | | activity | | |

Of the 133 CpG dinucleotides with gain of methylation, 71 known genes were identified. Applying high stringency, the functional cluster with the highest enrichment score included genes involved in pleckstrin homology, small domains that occur in a large variety of signaling proteins, where they serve as simple targeting domains that bind lipids. Methylation loss in 74 CpG dinucleotides was associated with 38 known genes. Signaling pathways involved endothelin signaling, T-cell activation, insulin signaling, progesterone-related oocyte maturation, MS-related immune injury, phospholipase C-epsilon, G-protein signaling (Gl, Gs), encephalin release and metabotropic glutamate receptor group 1 pathway in genes with significant methylation loss. Associated diseases included type II diabetes, Huntington disease and cystic fibrosis.

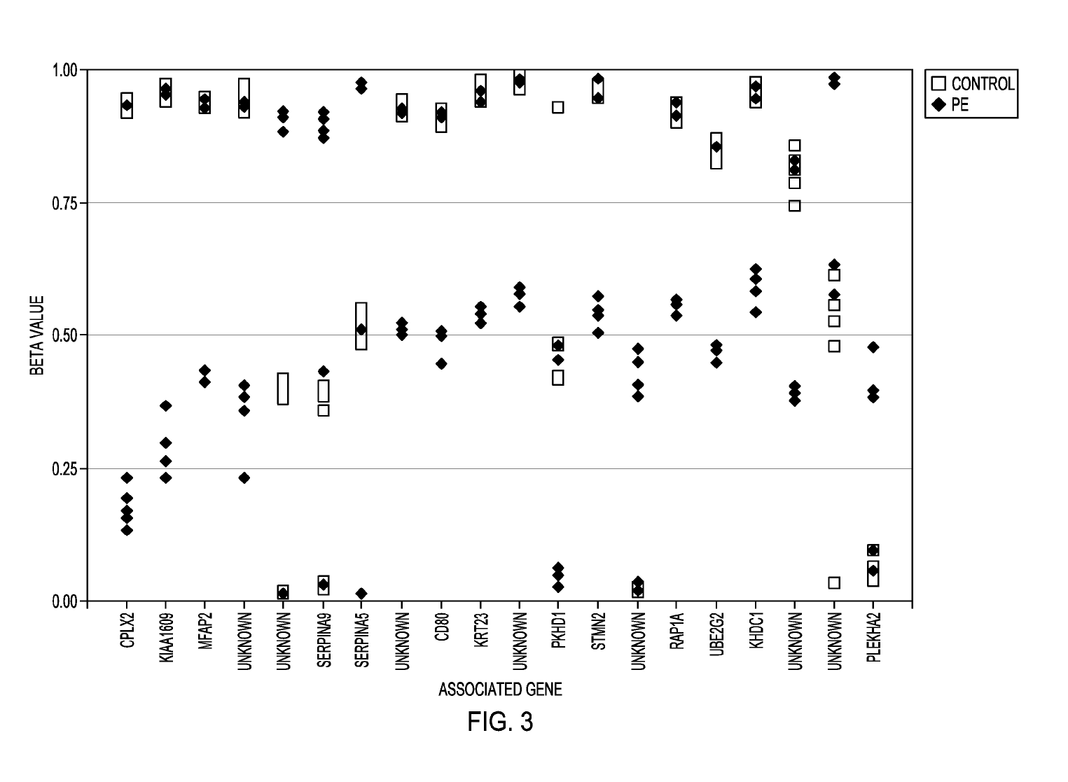

To further narrow focus to DNA methylation biomarkers that may have the greatest clinical utility, and to test whether this analysis can successfully identify CpG sites with potential clinical biomarker utility, additional Infinium methylation array analysis was performed using the NIMBL software package written using Matlab (Wessely & Emes, 2012). NIMBL identifies differentially methylated sites and corresponding genes between two groups of samples. The NIMBL data analysis tool also contains a module (NIMBL-qc) which allows for a quality assessment of samples. Briefly, multiple output plots are generated to visualize the sample quality. This analysis includes visualization of beta value distribution of each sample and measures deviation from the expected distribution as this is largely related to the detection pvalues. The identification of low quality samples is performed using a Kolmogorov-Smirnov test. All of the samples used in this study passed NIMBL QC, as did unmethylated and fully methylated control samples (data not shown). Differential methylation of top array sites is shown in FIG. 3. Twenty genes were identified as putative biomarkers for early screening of preeclampsia of which 13 were identifiable (Table 3). Of the 13 known genes identified by NIMBL analysis, 10 met the initial delta.beta criteria and 12 genes had significant differences in mean methylation in maternal white blood cells.

TABLE 3
Potential Early Screening Biomarkers in Preeclampsia

| | | | | | | | MPBC | Placenta |
|---|---|---|---|---|---|---|---|---|
| Gene | | | Phenotype | | Relationship | Relationship | Methylation | Methylation |
| Symbol | Gene Name | Gene Function | Associations | Chr | to Gene | to CpG Island | Delta Beta | Delta Beta |
| CPLX2 | Complexin 2 | Involved in | Susceptibility | 5 | TSS1500, | N_Shore | −0.51*** | 0.04 |
| | | synaptic vesicle | with | | 5′UTR | | | |
| | | exocytosis, | schizophrenia | | | | | |
| | | binding syntax in | | | | | | |
| | | (part of the SNAP | | | | | | |
| | | receptor), | | | | | | |
| | | causing | | | | | | |
| | | transmitter | | | | | | |
| | | release; involved | | | | | | |
| | | in mast cell | | | | | | |
| | | exocytosis | | | | | | |
| KIAA1609 | TBC/LysM- | Protein binding | | 16 | Body | | −0.34** | −0.03 |
| | associated domain | | | | | | | |
| | containing 1 | | | | | | | |
| | (TLDC1) | | | | | | | |
| MFAP2 | Microfibrillar- | Major antigen of | Susceptibility | 1 | TSS1500, | S_Shelf | −0.27** | −0.01 |
| | associated | elastin- | to severe | | TSS1500 | | | |
| | protein 2 | associated | congenital | | | | | |
| | | microfibrils; | contractural | | | | | |
| | | involved in | arachnodactyly | | | | | |
| | | etiology of | and obesity | | | | | |
| | | inherited | | | | | | |
| | | connective tissue | | | | | | |
| | | diseases | | | | | | |
| SERPINA9 | Serpin peptidase | Protease | Overexpressed | 14 | TSS1500, | | 0.33* | 0.33*** |
| | inhibitor, clade A | inhibitor; | in most | | TSS1500 | | | |
| | (alpha-1 | involved in | follicular | | | | | |
| | antiproteinase, | trypsin and | lymphomas | | | | | |
| | antitrypsin) | trypsin-like | | | | | | |
| | member 9 | serine protease | | | | | | |
| | | inhibition; | | | | | | |
| | | inhibits plasmin | | | | | | |
| | | and thrombin | | | | | | |
| *SERPINA5 | Serpin peptidase | Protease | Protein C | 14 | TSS1500 | | 0.18 | 0.02 |
| | inhibitor, clade A | inhibitor; | inhibitor | | | | | |
| | (alpha-1 | involved in | (plasminogen | | | | | |
| | antiproteinase, | trypsin and | activator | | | | | |
| | antitrypsin) | trypsin-like | inhibitor-3) | | | | | |
| | member 5 | serine protease | | | | | | |
| | | inhibition; | | | | | | |
| | | involved in | | | | | | |
| | | hemostasis and | | | | | | |
| | | thrombosis | | | | | | |
| | | through | | | | | | |
| | | inhibition of | | | | | | |
| | | serine proteases | | | | | | |
| | | protein C, | | | | | | |
| | | plasminogen | | | | | | |
| | | activators and | | | | | | |
| | | kallikreins | | | | | | |
| CD80 | Cluster of | Encodes protein | Putative | 3 | TSS1500 | | −0.21** | 0.02 |
| | differentiation | activated by | susceptibility | | | | | |
| | (CD) 80 molecule | binding of CD28 | gene for | | | | | |
| | | to CTLA-4, | rheumatoid | | | | | |
| | | inducing T-cell | arthritis and | | | | | |
| | | proliferation and | SLE | | | | | |
| | | cytokine | | | | | | |
| | | production | | | | | | |
| KRT23 | Keratin 23 | Intermediate | None | 17 | TSS1500 | | −0.24** | −0.08 |
| | (histone | filaments | indentified | | | | | |
| | deacetylase | responsible for | | | | | | |
| | inducible) | structrual | | | | | | |
| | | integrity of | | | | | | |
| | | epithelial cells | | | | | | |
| PKHD1 | Polycystic kidney | Involved in | Polycystic | 6 | TSS1500, | | −0.30** | −0.14 |
| | and hepatic | bipolar cell | kidney | | TSS1500 | | | |
| | disease 1 | division through | disease, | | | | | |
| | (autosomal | regulation of | infantile type | | | | | |
| | recessive) | centrosome | with hepatic | | | | | |
| | | duplication and | fibrosis 1 | | | | | |
| | | mitotic spindle | (ARPKD) | | | | | |
| | | assembly; | | | | | | |
| | | receptor protein | | | | | | |
| | | involved in | | | | | | |
| | | collecting duct | | | | | | |
| | | and biliary | | | | | | |
| | | differentiation | | | | | | |
| STMN2 | Stathmin-like 2 | Encodes proteins | | 8 | Body | | −0.23** | 0.08 |
| | | for microtubule | | | | | | |
| | | dynamics and | | | | | | |
| | | signal | | | | | | |
| | | transduction; | | | | | | |
| | | regulatory role in | | | | | | |
| | | neuronal growth; | | | | | | |
| | | decreases | | | | | | |
| | | expression | | | | | | |
| | | associated with | | | | | | |
| | | AD and Down | | | | | | |
| | | Syndrome | | | | | | |
| *RAP1A | RAP1A, member | Encodes protein | | 1 | TSS1500 | N. Shore | −0.19** | −0.007 |
| | of RAS oncogene | counteracting | | | | | | |
| | family | mitogenic | | | | | | |
| | | function of RAS; | | | | | | |
| | | involved in | | | | | | |
| | | maintenance of | | | | | | |
| | | endothelial | | | | | | |
| | | junction integrity | | | | | | |
| *UBE2G2 | Ubiquitin- | Involved in | | 21 | 5′UTR, Body | N. Shore | −0.18** | −0.08 |
| | conjugating | targeting | | | | | | |
| | enzyme E2G 2 | proteins for | | | | | | |
| | | degradation | | | | | | |
| KHDC1 | KH homology | Member of | Unknown | 6 | TSS1500 | S_Shore | −0.20** | 0.12 |
| | domain | oocyte and/or | | | | | | |
| | containing 1 | embryonic stem | | | | | | |
| | | cell-specific | | | | | | |
| | | genes | | | | | | |
| PLEKHA2 | Pleckstrin | Binds to | Unknown | 8 | 5′UTR | Island | 0.22* | −0.04 |
| | homology domain | phosphatidylinositol | | | | | | |
| | containing, | 3,4-diphosphate | | | | | | |
| | family A | | | | | | | |
| | (phosphoinositide | | | | | | | |
| | binding specific) | | | | | | | |
| | member 2 | | | | | | | |

MPBC, maternal peripheral white blood cells
Delta beta, CpG methylation reported as difference in preeclampsia compared to control [symbol]
Genes not meeting pre-established delta beta of ≧0.2 or ≦−0.02
*= p < 0.05;
**= p < 0.01;
***= p < 0.001 [symbol]
indicates data missing or illegible when filed

Interpretation of DNA methylation data and evaluation of potential biomarkers is complex due to the heterogeneity of primary clinical samples. The NIMBL platform allows for masking of a limited number of samples to allow for biomarker identification despite heterogeneity amongst samples. This can be seen in FIG. 3 where one or two preeclampsia samples may cluster with normotensive controls for each spot tested, despite the statistically significant differences in average beta score between the two groups and the clear separation of the majority of preeclampsia samples from control in terms of potentially informative sites. These data underscore the need to perform this type of secondary analysis and not simply rely on average beta scores, as it is clear from this analysis that all individual patients are not expected to have 100% of the abnormal biomarkers despite statistically significant differences in beta-scores between the two groups. We expect that the most successful and clinically evaluable DNA methylation biomarker panels will need to include multiple

DNA methylation sites and will rely on clinically evaluable distance in average beta that distinguishes preeclampsia from normotensive pregnancy.

Transmission of DNA Methylation Biomarkers of Preeclampsia from Mother to Offspring During Pregnancy

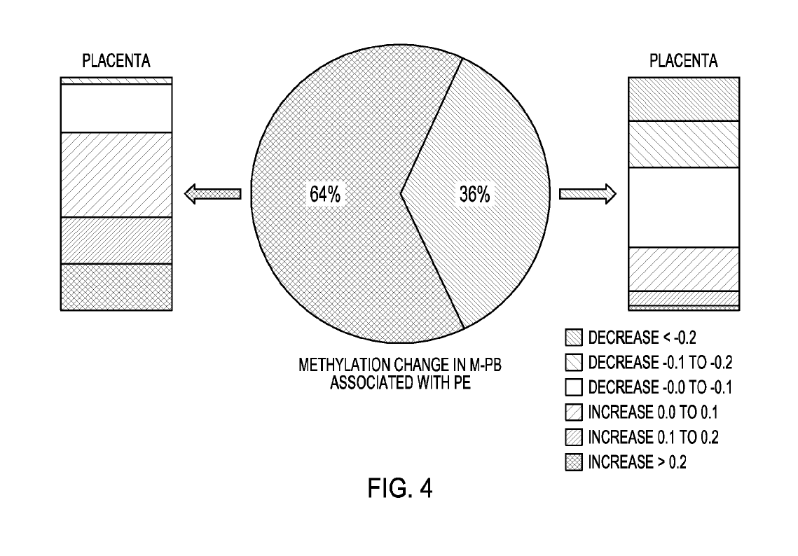

Finally, it was tested whether DNA methylation across the genome may play a role in facilitating the transgenerational transmission of preeclampsia risk from parent to offspring. In order to begin to assess whether DNA methylation changes are also seen in offspring of women with preeclampsia, DNA was isolated from the placenta at time of delivery and measured global methylation changes using the Illumina Infinium platform as described above. CpG dinucleotides identified as being differentially methylated in MPBCs (FIG. 2) were analyzed for methylation changes in placental tissue from women with preeclampsia as compared to normotensive controls (FIG. 4). The majority of the 64% of differentially methylated sites in MPBCs that were associated with a significant gain of methylation also showed varying amounts of methylation gain in placenta tissue with many sites showing significant methylation gains in placenta based on a delta.beta >0.2 (FIG. 4; left placental panel). Further, 36% of differentially methylated sites in MPBCs that were associated with a significant loss of methylation were likely to also show a loss of methylation to varying degrees in placental tissue with many sites showing significant methylation losses based on a delta.beta <−0.2 (FIG. 4; right panel).

### Discussion

Provided here is data demonstrating for differential DNA methylation in pregnancies complicated by preeclampsia. Genome-wide DNA methylation was quantified in individual CpG sites, identifying significant differences in maternal white blood cells and placenta from women who developed preeclampsia compared to those with normotensive pregnancies. Genome-wide DNA methylation data showed clear separation between samples from women with preeclampsia and those with normotension for many individual sites. Differences in DNA methylation were identified in maternal white blood cells collected during the first trimester in women who subsequently developed preeclampsia, compared to those women with a normotensive pregnancy. Currently preeclampsia cannot be diagnosed until later in pregnancy when women manifest the characteristic signs and symptoms, a point at which significant systemic injury has been established. A biologically sound mechanistic explanation of how preeclampsia develops has not been identified. This gap in knowledge hinders the development of a reliable method appropriate for early screening and diagnosis in the clinical setting. Current methods currently employed include: sonographic placental maturation (Walker, Hindmarsh, Geary, & Kingdom, 2010), ultrasound uterine artery Doppler pulsatility indices (Plasencia et al., 2010; Stampalija, Gyte, & Alfirevic, 2010), serum markers (Martin & Brown, 2010; Wortelboer et al., 2010) and identification of clinical indicators. Blood pressure exceeding 140 mmHg systolic or 90 mmHg diastolic with significant proteinuria of >300mg/24 hours are used to diagnose preeclampsia after significant pathology has developed leading to clinical manifestations. In this study, maternal blood samples were obtained in early pregnancy prior to the diagnosis of preeclampsia in all cases and represents DNA methylation biomarkers that can be used for early detection and intervention strategies.

The NIMBL method is useful for the development of a DNA methylation biomarker panel with either increased or decreased methylation in preeclampsia that can be used for early detection of preeclampsia in maternal peripheral blood, as early as the first trimester. The specific biomarkers identified by the NIMBL method have not been previously reported in the preeclampsia literature. However, several of the genes may play a role in pregnancy, miscarriage, implantation, or immune tolerance. Stathmin family and KH homology domain genes play a role in pregnancy and implantation (Schulz, Widmaier, Qiu, & Roberts, 2009; Tian, Pascal, Fouchecourt, Pontarotti, & Monget, 2009). The E2 class of ubiquitin-conjugating enzyme is upregulated in early miscarriage (A. X. Liu et al., 2006). CD80 plays a role in maternal immune tolerance to the fetus (Abumaree, Chamley, Badri, & El-Muzaini, 2012; Moldenhauer, Keenihan, Hayball, & Robertson, 2010) Lastly, RAP1A protein was noted to be down regulated in gestational diabetes mellitus (B. Liu et al., 2012). Additionally, some of the biomarkers disclosed herein have other members of their gene families reported to be differentially expressed in preeclampsia. For example, the SERPIN family, and in particular SERPINA3, have been implicated in preeclampsia (Blumenstein et al., 2009). However, there are no reports of differential expression or regulation of SERPINA5 or SERPINA9 in preeclampsia. It is reported herein, for the first time, differential methylation of genes in maternal white blood cells and placental tissue in preeclampsia. Development of a selective and specific biomarker panel can identify individuals at increased risk of developing preeclampsia who may benefit from early intervention strategies. Further, screening prior to pregnancy in both males and females may be used to predict future risk of preeclampsia-complicated pregnancy.

Analysis of DNA methylation in fetal-derived placental tissue was also clearly different when comparing tissue from normotensive pregnancies and pregnancies complicated by preeclampsia, suggesting that DNA methylation changes can be propagated across generations. Other investigators have identified differential methylation in single genes in either placental tissue or maternal sera, though the finding of differentially methylated genes common to both mothers and fetal tissue have not been reported. Willer et al. (2004) reported a significantly higher percentage of differential methylation in the APC gene from cell-free DNA in sera of women during the first trimester who subsequently developed preeclampsia (Muller et al., 2004). As both maternal and fetal cell free DNA has been reported in maternal sera, the source of the DNA (maternal vs. fetal) could not be definitively determined The APC gene is involved in pathways that counter metastasis. Increased methylation in early pregnancy among women who developed preeclampsia may be associated with the impaired trophoblast invasion that occurs during placental development in preeclampsia, leading to placental insufficiency. While methylation differences are significant in isolation, that common differences were identified in maternal blood taken in the first trimester of pregnancy and in fetal-derived placental tissue is a novel finding that has the potential to change clinical practice, improving health outcomes for mothers and their children.

### Bibliography

Anderson, C. M. (2007). J Obstet Gynecol Neonatal Nurs, 36(1), 3-8.

Anderson, C. M., et al. (2005a). Am J Obstet Gynecol, 193(1), 267-72.

Anderson, C. M., et al. (2005b). Biol Reprod, 72(3), 762-6.

Anderson, C. M., et al. (2006). Exp Biol Med (Maywood), 231(8), 1398-402.

Anderson, C. M., et al. (2006). Biol Reprod, 74(3), 538-44.

Arngrimsson, R. (2005). Nat Genet, 37(5), 460-1.

Barton, J. R., & Sibai, B. M. (2008). Obstetrics and Gynecology, 112(2 Pt 1), 359-372. doi: 10.1097/AOG.0b013e3181801d56

Berends, A. L., et al. (2008). Hypertension, 51(4), 1034-41.

Bibikova, M., et al. (2011). Genomics, 98(4), 288-295. doi: 10.1016/j.ygeno.2011.07.007; 10.1016/j.ygeno.2011.07.007

Carty, D. M., et al. (2010). Journal of Hypertension, 28(7), 1349-1355. doi: 10.1097/HJH.0b013e32833a39d0

Chappell, S., & Morgan, L. (2006). Clinical Science (London, England : 1979), 110(4), 443-458. doi: 10.1042/CS20050323

Conde-Agudelo, A., et al. (2004). Obstetrics and Gynecology, 104(6), 1367-1391. doi: 10.1097/01.AOG.0000147599.47713.5d

Dedeurwaerder, S., et al. (2011). Epigenomics, 3(6), 771-784. doi: 10.2217/epi.11.105; 10.2217/epi.11.105

Doi, A., et al. (2009). Nature Genetics, 41(12), 1350-1353. doi: 10.1038/ng.471; 10.1038/ng.471

Donkena, K. V., et al. (2010). Obstetrics and Gynecology International, 2010, 302051. doi:10.1155/2010/302051

Esplin, M. S., et al. (2001). The New England Journal of Medicine, 344(12), 867-872.

Fabry, I. G., et al. (2010). Acta Clinica Belgica, 65(4), 229-236.

Geelhoed, J. J., et al. (2010). Circulation, 122(12), 1192-1199. doi: 10.1161/CIRCULATIONAHA.110.936674

Ghulmiyyah, L., & Sibai, B. (2012). Seminars in Perinatology, 36(1), 56-59. doi: 10.1053/j.semperi.2011.09.011

Gruslin, A., & Lemyre, B. (2011a). Best Practice & Research.Clinical Obstetrics & Gynaecology, 25(4), 491-507. doi:10.1016/j.bpobgyn.2011.02.004; 10.1016/j.bpobgyn.2011.02.004

Gruslin, A., & Lemyre, B. (2011b). Best Practice Research.Clinical Obstetrics Gynaecology, 25(4), 491-507. doi: 10.1016/j.bpobgyn.2011.02.004

Hadker, N., et al. (2010). Journal of Medical Economics, 13(4), 728-737. doi: 10.3111/13696998.2010.535285

Harskamp, R. E., & Zeeman, G. G. (2007). Am J Med Sci, 334(4), 291-5. Herceg, Z. (2007). Mutagenesis, 22(2), 91-103. doi: 10.1093/mutage/ge1068

Hutcheon, J., et al. (2011). Best Practice & Research.Clinical Obstetrics Gynaecology, 25(4), 391-403. doi: 10.1016/j.bpobgyn.2011.01.006

Irizarry, R. A., et al. (2009). Official Journal of the International Mammalian Genome Society, 20(9-10), 674-680. doi: 10.1007/s00335-009-9222-5; 10.1007/s00335-009-9222-5

Jia, R. Z., et al. (2012). International Journal of Molecular Medicine, 30(1), 133-141. doi: 10.3892/ijmm.2012.983; 10.3892/ijmm.2012.983

Jirtle, R. L., & Skinner, M. K. (2007). Nature Reviews.Genetics, 8(4), 253-262. doi: 10.1038/nrg2045

Jones, P. A., & Baylin, S. B. (2007). Cell, 128(4), 683-692. doi:10.1016/j.ce11.2007.01.029

Klose, R. J., & Bird, A. P. (2006). Trends in Biochemical Sciences, 31(2), 89-97. doi: 10.1016/j.tibs.2005.12.008

Kulkarni, A., et al. (2011). DNA and Cell Biology, 30(2), 79-84. doi: 10.1089/dna.2010.1084

Lahiri, D. K., et al. (2009). Molecular Psychiatry, 14(11), 992- 1003. doi: 10.1038/mp.2009.82

Lampinen, K. H., et al. (2006). J Hypertens, 24(4), 751-6.

Lillycrop, K. A., et al. (2005). J Nutr, 135(6), 1382-6.

Lofton Day, et al. (2008). Clinical Chemistry, 54(2), 414-423. doi: 10.1373/clinchem.2007.095992

Loke, Y., et al. (2012). Twin Research and Human Genetics, 1-8. doi: 10.1017/thg.2012.114

Mangos, G. J. (2006). J Hypertens, 24(4), 639-41.

Marin, R., et al. (2000). Hypertens Pregnancy, 19(2), 199-209.

Martin, A. C., & Brown, M. A. (2010). Nature Reviews.Nephrology, 6(12), 744-748. doi: 10.1038/nrneph.2010.125

Muller, H. M., et al. (2004). Clinical Chemistry, 50(6), 1065-1068. doi: 10.1373/clinchem.2003.030387

Ohm, J. E., et al. (2010). Cancer Research, 70(19), 7662-7673. doi: 10.1158/0008-5472.CAN-10-1361

Plasencia, W., et al. (2010). Official Journal of the International Society for the Study of Hypertension in Pregnancy, doi: 10.3109/10641955.2010.506232

Qureshi, I. A., & Mehler, M. F. (2010). Archives of Neurology, 67(11), 1316-1322. doi: 10.1001/archneuro1.2010.275

Ray, J. G., et al. (2005). Lancet, 366(9499), 1797-803.

Ren, J., et al. (2011). Cellular Signalling, 23(7), 1082-1093. doi: 10.1016/j.cellsig.2011.02.003

Roberts, J. M., & Hubel, C. A. (2010). Official Publication of the Jacobs Institute of Women's Health, 20(5), 304-307. doi: 10.1016/j.whi.2010.05.004

Roberts, J. M. et al. (2003). Hypertension, 41(3), 437-45.

Sandoval, J., et al. (2011). Official Journal of the DNA Methylation Society, 6(6), 692-702.

Skjaerven, R., et al. (2005). Bmj, 331(7521), 877.

Smith, G. C., et al. (2001). Lancet, 357(9273), 2002-6.

Staff, A. C., et al. (2010). Hypertension, 56(6), 1026-1034. doi: 10.1161/HYPERTENSIONAHA.110.157743

Stampalija, T., et al. (2010). Cochrane Database of Systematic Reviews (Online), (9)(9),CD008363. doi: 10.1002/14651858.CD008363.pub2

Tunc, O., & Tremellen, K. (2009). Journal of Assisted Reproduction and Genetics, 26(9-10), 537-544. doi: 10.1007/s10815-009-9346-2

Walker, M. G., et al. (2010). Journal of Obstetrics and Gynaecology Canada: JOGC=Journal d'Obstetrique Et Gynecologie Du Canada : JOGC, 32(12), 1134-1139.

Wang, X., et al. (2011). Current Hypertension Reports, 13(6), 442-451. doi: 10.1007/s11906-011-0230-y

Weaver, I. C., et al. (2004). Annals of the New York Academy of Sciences, 1024, 182-212. doi: 10.1196/annals.1321.099

Weber, B., et al. (2007). Cell Cycle (Georgetown, Tex.), 6(9), 1001-1005.

Wessely, F., & Emes, R. D. (2012). Frontiers in Genetics, 3, 161. doi: 10.3389/fgene.2012.00161; 10.3389/fgene.2012.00161

Williams, P., & Broughton Pipkin, F. (2011). Best Practice Research. Clinical Obstetrics Gynaecology, 25(4), 405-417. doi: 10.1016/j.bpobgyn.2011.02.007

Wilson, B. J., et al. (2003). BMJ, 326(7394), 845.

Wortelboer, E. J, et al. (2010). BJOG: An International Journal of Obstetrics and Gynaecology, 117(11), 1384-1389. doi: 10.1111/j.1471-0528.2010.02690.x; 10.1111/j.1471-0528.2010.02690.x

Xiao, Q., & Ye, S. (2011). The genetics of epigenetics: Is there a link with cardiovascular disease. Heart (British Cardiac Society), 97(2), 96-97. doi: 10.1136/hrt.2010.214833

Yuen, R. K., et al. (2010). European Journal of Human Genetics: EJHG, doi: 10.1038/ejhg.2010.63

Zawia, N. H., et al. (2009). Free Radical Biology & Medicine, 46(9), 1241-1249. doi: 10.1016/j.freeradbiomed.2009.02.006

All publications, nucleotide and amino acid sequence identified by their accession nos., patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims. As used herein and in the appended claims, the singular forms “a,” “an,” and “the” include plural reference unless the context clearly dictates otherwise. Thus, for example, a reference to “a nucleic acid” or “a polypeptide” includes a plurality of such nucleic acids or polypeptides (for example, a solution of nucleic acids or polypeptides or a series of nucleic acid or polypeptide preparations), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein.

Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

1. A method to diagnose preeclampsia in a patient comprising measuring methylation level of at least one gene selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 in a first biological sample from a subject; and diagnosing preeclampsia in said subject based on a higher or lower methylation level in the first biological sample relative to the methylation level in a second biological sample from an individual without preeclampsia or control.

2. The method of 1, wherein the methylation level of at least two genes selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 and any combination thereof is measured.

3. The method of claim 2, wherein the methylation level of at least 3 genes selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 and any combination thereof is measured.

4. The method of claim 3, wherein the methylation level of at least 4 genes selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 and any combination thereof is measured.

5. The method of claim 4, wherein the methylation level of at least 5 genes selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 and any combination thereof, is measured.

6. The method of claim 5, wherein the methylation level of at least 6, 7, 8, 9, 10, 11, 12 or 13 genes selected from the group consisting of CPLX2, KIAA1609, MFAP2, CD80, KRT23, PKHD1, STMN2, RAP1A, UBE2G2, KHDC1, SERPINA9, SERPINA5 and PLEKHA2 and any combination thereof, is measured.

7. The method of claim 1, wherein an increased level of methylation of at least one gene selected from the group consisting of SERPINA9, SERPINA5 and PLEKHA2 indicates that the subject has or will develop preeclampsia.

8. The method claim 1, wherein a decreased level of methylation of least one gene selected from the group consisting of CPLX2, KIAA1609, MFAP2, CD80, KRT23, PKHD1, STMN2, RAP1A, UBE2G2 and KHDC1 indicates that the subject has or will develop preeclampsia.

9. The method of claim 1, wherein the subject and individual are mammalian

10. The method of claim 9, wherein the mammal is human.

11. The method claim 1, wherein the first and second samples comprise blood.

12. The method of claim 1, further comprising treating the subject for preeclampsia.
